(19) United States
(12) Patent Application Publication
(10) Pub. No.: US 2011/0262370 A1
(43) Pub. Date: Oct. 27, 2011

(54) Shave Preparations

(76) Inventors: Kenneth Eugene Kyte, III, Oregonia, OH (US); Timothy Woodrow Coffindaffer, Maineville, OH (US)

(21) Appl. No.: 13/095,343

(22) Filed: Apr. 27, 2011

Related U.S. Application Data
(60) Provisional application No. 61/328,612, filed on Apr. 27, 2010.

(51) Int. Cl. A61K 8/02; A61Q 9/02

(52) U.S. Cl. 424/47

(57) ABSTRACT

The shave preparation comprising water; one or more lipophilic skin conditioning agents; one or more thickening agents; one or more emulsifying agents; and one or more lubricants; a volatile self-foaming agent, wherein the composition is in the form of a self-foaming cream or lotion having an elastic modulus (G′) of about 150 Pa to about 1000 Pa having a low level of anionic surfactants and/or soap.

### CROSS REFERENCE TO RELATED APPLICATIONS

This patent claims priority to U.S. Provisional Application No. 61/328,612 filed Apr. 27, 2010.

### FIELD OF THE INVENTION

The present invention generally relates to a shave preparation which is preferably self-foaming in the form of a cream and/or lotion.

### BACKGROUND OF THE INVENTION

Various shave preparations may be found in Harry's Cosmeticology, Seventh Edition, J. B. Wilkinson and R. J. Moore (editors), Chemical Publishing, New York, 1982, pp. 156-189. Currently, one of the most widely used forms of shave preparations are the types referred to as instant foams and self-foaming gels (also known as post-foaming gels). Instant foams comprise water, a dispersed or solubilized surface-active agent (e.g. a soap, an anionic, cationic, amphoteric, or nonionic surface-active agent, or a combination of these agents), and a propellant/blowing agent. These types of compositions are often dispensed from pressurized aerosol containers in the form of a rich foam lather for spreading by hand on the area to be shaved. Post-foaming gels often include many of the same ingredients as instant foams. These are propellant-in-water emulsions that are formulated as thick, stiff gels which have incorporated into the composition a small amount of blowing agent (typically a volatile hydrocarbon). When the gel is rubbed onto a warm surface, like skin, the blowing agent volatilizes and causes the gel to turn into a foam lather.

Examples of known shave preparations are also disclosed in U.S. Pat. No. 6,682,726, to Marchesi et al. This reference discloses a self-foaming lotion having an elastic modulus (G′) of about 100 Pascals to about 1000 Pascals. Another known shave preparation is provided in WO Pub. 94/02109 A2 to Monson et al. This reference discloses a number of non-soap compositions which have been found to be runny in nature when applied to the hand or face directly. The Monson formulations are self described as being designed to foam instantaneously on spreading with a single motion and develop into copious foam.

Despite these and other attempts, there remains a need for a self-foaming shave preparation which does not include a harsh or potentially skin irritating surfactant system, yet has the desired elastic modulus to provide improved rinsability and spreading/dispersing.

### SUMMARY OF THE INVENTION

One aspect of the invention provides for a self-foaming shave preparation in the form of a cream or lotion. The shave preparation comprises water; one or more lipophilic skin conditioning agents; one or more thickening agents; one or more emulsifying agents; and one or more lubricants; a volatile self-foaming agent, wherein the composition is in the form of a self-foaming cream or lotion having an elastic modulus (G′) of at least about 150 Pa to about 1000 Pa, preferably between about 175 Pa to about 800 Pa, most preferably between about 175 Pa and 600 Pa (measured with a rheometer [e.g. AR-2000, TA Instruments, 4 cm acrylic plate] at a frequency of 1 Hz, oscillatory stress range 0.01 to 1.0 Pa, temperature 5° C., Gap 1000 um).

In one embodiment, the shave preparation contains less than about 2% anionic surfactant and/or soap, more preferably less than about 1% anionic surfactant, most preferably less than about 0.5% anionic surfactant, optionally even being free or substantially free of said anionic surfactant and/or soap. Another aspect of the invention provides for the shave preparation described above, wherein the shave preparation is free or substantially free of a gel network. Another aspect of the invention provides for the shave preparation described above, further comprising a density reducing agent.

Yet another aspect of the invention provides for a self-foaming shave preparation in the form of a cream or lotion. The shave preparation comprises: at least 60% water; from about 2% to about 4% of one or more lipophilic skin conditioning agents; from about 0.25% to about 3% of one or more thickening agents; from about 0.25% to about 3% of one or more emulsifying agents; and from about 0.25% to about 3% of one or more lubricants; a volatile self-foaming agent, wherein the composition is in the form of a self-foaming cream or lotion having an elastic modulus (G′) of about 150 Pa to about 1000 Pa, preferably between about 175 Pa to about 800 Pa, most preferably between about 175 Pa and 600 Pa (measured with a rheometer [e.g. AR-2000, TA Instruments, 4 cm acrylic plate] at a frequency of 1 Hz, oscillatory stress range 0.01 to 1.0 Pa, temperature 5° C., Gap 1000 um).

### DETAILED DESCRIPTION OF THE INVENTION

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

The current invention provides for shave preparations having desirable properties that improve the shave experience. Without wishing to be bound by theory, it is believed that the compositions within the shave care preparations of the present invention interact synergistically to provide for an enhanced experience.

### Water

The shave preparations of the current invention comprise water. In one embodiment, the shave preparation comprises at least about 60% by weight water. In an alternate embodiment, the shave preparation comprises at least about 70% by weight water. In an alternate embodiment, the shave preparation comprises at least about 80% by weight water. In an alternate embodiment, the shave preparation comprises at least about 85% by weight water.

### Lipophilic Skin Conditioning Agent

Shave preparations of the present invention employ one or more lipophilic skin conditioning agents. The concentration level of the skin conditioning agents either singularly or collectively may range from about 1% to about 12% by weight of the base composition. Some preferred concentration levels include greater than about 1%, from about 2% to about 5%, and from about 2% to about 4%. It is to be understood that the scope of appended claims that do not specify a concentration level of the lipophilic skin conditioning agent is not limited to the levels described in this paragraph.

Exemplary skin conditioning agents include hydrocarbons, polymeric hydrocarbons, esters, ethers, and silicones selected from the group consisting of alkyl ethers, mineral oil, isoparaffin, greater than C20 hydrogenated polyisobutene, and petrolatum; and an ester composed of a linear or branched C16-C22 alkyl chain and a mono alkyl group consisting of a linear or branched C1 to C6 alkyl chain. Some preferred skin conditioning agents comprise isostearic acid derivatives; for example, isostearyl isostearate, isopropyl isostearate, and mixtures thereof, PPG-15 Stearyl Ether and dimethicone. Other skin conditioning agents known to the skilled artisan may also be employed depending on the form of the personal care composition and the targeted skin benefit.

The skin conditioning agents may also help to reduce the coefficient of friction for personal care compositions provided herein that are in the form of shaving compositions. The reduction in friction can decrease the potential for skin irritation that can arise from contacting the skin one or more times with a razor blade. Employment of the skin conditioning agent in this context may also permit formulation flexibility regarding the type and concentration level of lubricants (as discussed more fully below) that are included in the shaving preparations.

In one embodiment, the shave preparations of the present invention is free or substantially free of any cationic skin conditioning agents, including but not limited to cationic polymers and cationic ammonium salts. Examples of such skin conditioning agents include substituted quaternary ammonium compounds (i.e., quaterniums, stearalkonium chloride, and guar hydroxypropytrimonium chloride.) See U.S. Pat. No. 4,586,650 at col. 2 line 54 et seq for additional examples of cationic skin conditioning agents which are preferably limited or not used in the present invention Importantly, the present invention is able to achieve a desirable shave experience without requiring said cationic skin conditioning agents. This has been found to provide desired shave performance while reducing formulation costs and complexity. As defined herein, “substantially free” means that no amount of said ingredients intentionally added into the composition, but allowing for trace amounts which may be carried over from other ingredients or from processing conditions.

### Thickening Agent (Including Thickeners and Gelling Agents)

The shave preparations of the present invention contain one or more thickening agents suitable for thickening the composition to a cream or lotion form having the desired elastic modulus, from about 0.1% to about 5%, alternatively from about 0.1% to about 4%, alternatively from about 0.25% to about 3%, by weight of the composition.

The preferred rheological property by which the creams or lotions of the present invention may be defined is elastic modulus. The self-foaming creams or lotions will typically have an elastic modulus (G′) of between about 150 Pa to about 1000 Pa, preferably between about 175 Pa to about 800 Pa, most preferably between 200 Pa and about 600 Pa (measured with a Prep Rheometry Test Method. In one embodiment, the shave preparation has an elastic modulus of from about 150 Pa to about 550 Pa, or from about 200 Pa to about 420 Pa, or from about 220 Pa to about 300 Pa, as measured according to the Prep Rheometry Test Method.

Prep Rheology Test Method:

Elastic Modulus (G′) measurements are performed via Stress Sweep using an AR2000 Rheometer (TA Instruments) with a temperature controlled peltier and a 40 mm acrylic parallel plate.
1. Before performing the measurement for the first time, the zero gap is set and rotational mapping was performed.
2. The temperature of the peltier is reduced to 5 degrees C. and a volume of the sample composition, sufficient to cover the upper surface of the acrylic plate, is dispensed directly onto the surface, centered under the acrylic plate. All subsequent measurements are made at 5 degrees C.
3. The acrylic plate is then lowered to a gap of 1000 microns. The excess product is scraped off from around the edges of the acrylic plate using a straight edged piece of plastic and any product on the top of the acrylic plate is wiped off.
4. The sample was allowed to equilibrate for 15 seconds before the first measurement is taken. The frequency is set to 1 Hz and the oscillatory stress is varied from 0.01 Pa to 1 Pa.
5. Once the program has completed running, the Elastic Modulus (G′) measurements are taken over the oscillatory stress range mentioned above. These measurements are then averaged together and reported.

Without intending to be bound by theory, it has been found that the shave preparation of the present invention has a desired elastic modulus to provide improved rinsability and spreading/dispersing. It has been found that shave preparations with elastic modulus being below 150 Pa were found to be undesirably runny when dispensed and difficult to control when applying the product to a vertical surface such as the face.

In one embodiment, the shave preparation has the above elastic modulus without the need for a gel network composition. Importantly, it is believed that other attempts to provide shave preparations of desirable elastic modulus have relied on the addition of gel network compositions. As such, in one embodiment, the present shave preparation is free or essentially free of a gel network. To provide a desirable elastic modulus, it is believed that a minimum amount of thickener is needed, for example at least from about 0.1% to about 5%.

Nonlimiting classes of thickening agents include those selected from the following: Carboxylic Acid Polymers, Crosslinked Polyacrylate Polymers Polyacrylamide Polymers, Polysaccharides, Clays and Gums, and mixtures thereof when appropriate.

In one embodiment, compositions of the present invention include a thickening agent selected from carboxylic acid polymers, crosslinked polyacrylate polymers, polyacrylamide polymers, polysaccharides, and mixtures thereof, more preferably selected from carboxylic acid polymers, polyacrylamide polymers, polysaccharides, and mixtures thereof.

### Emulsifier

The shave preparations of the present invention contain one or more emulsifying agents, from about 0.1% to about 8%, alternatively from about 0.1% to about 5%, alternatively from about 0.25% to about 3%, by weight of the composition.

Nonlimiting examples of surfactants for emulsification for use in the compositions of the present invention are disclosed in McCutcheon's, Detergents and Emulsifiers, North American edition (1986), published by allured Publishing Corporation; and McCutcheon's, Functional Materials, North American Edition (1992). Preferred emulsifiers are nonionic surfactants/emulsifiers. Nonlimiting useful emulsifiers herein include those selected from the group consisting of alkyl glucosides, alkyl polyglucosides, polyhydroxy fatty acid amides, alkoxylated fatty acid esters, sucrose esters, alkoxylated fatty alcohols, amine oxides, and mixtures thereof.

### Lubricants

Shave compositions of the present invention may employ one or more lubricants, from about 0.1% to about 8%, alternatively from about 0.1% to about 5%, alternatively from about 0.25% to about 3%, by weight of the composition.

Exemplary lubricants include lubricous water soluble polymers, water insoluble particles, and hydrogel-forming (or water swellable) polymers, and mixtures thereof.

Useful lubricious water soluble polymers may have a molecular weight greater between about 300,000 and 15,000,000 daltons, preferably more than about one million Daltons. Nonlimiting examples of suitable lubricious water soluble polymers include polyethylene oxide, polyvinylpyrrolidone, and polyacrylamide. Nonlimiting useful water insoluble particles may include inorganic particles or organic polymer particles. Hydrogel-forming polymers are typically highly hydrophilic polymers that, in water, form organized three-dimensional domains of approximately nanometer scale. Additional polymer lubricants include: cellulose derivatives such hydroxyalkyl cellulose polymers such as hydroxyethyl cellulose and hydroxypropyl cellulose, carboxymethyl cellulose, and cellulose methyl ether and polysaccharide gums such as, for example, xanthan gum, carrageenan gum, guar gum, locust bean gum, and hydroxypropyl guar gum.

### Gel Network

In one embodiment, the shave composition is substantially free from a gel network. As used herein, the term “gel network” refers to a lamellar or vesicular solid crystalline phase which comprises at least one fatty amphiphiles. In one embodiment, the present invention contains less than about 6%, alternatively less than about 3%, alternatively less than about 1%, alternatively less than about 0.5% of at least one fatty amphiphiles. Gel networks have been found to reduce the rinse profile of these systems. Fatty alcohol gel networks have been used for years in cosmetic creams and hair conditioners. Gel networks are a re-solidified liquid crystal gel phase formed by fatty amphiphiles (e.g. cetyl or stearyl alcohol) and a hydrophilic phase (e.g. water). It is formed by undergoing a melting and then re-solidification process in the hydrophilic phase. The gel network will typically have a lower thermal transition than the melt temperature of the fatty amphiphile itself.

### Post Foaming Agent

The post-foaming agent, when included in the post foaming gel composition, may be any volatile hydrocarbon or halohydrocarbon with a sufficiently low boiling point that it will volatilize and foam the gel upon application to the skin, but not so low that it causes the gel to foam prematurely. The typical boiling point of such an agent generally falls within the range of −20° to 40° C. Preferred post-foaming agents are selected from saturated aliphatic hydrocarbons having 4 to 6 carbon atoms, such as n-pentane, isopentane, neopentane, n-butane, isobutane, and mixtures thereof. Most preferred is a mixture of isopentane and isobutane in a weight ratio (IP:IB) of about 1:1 to about 9:1, preferably about 2:1 to about 7:1, most preferably about 3:1. The post-foaming agent will normally be selected so as to provide a vapor pressure at 20° C. of about 3 to about 20 psig, preferably about 5 to about 15 psig.

Density Reducing agents

Shave preparations of the present invention may also include materials for reducing the density of the product in order to enhance the feeling of protection from the razor. Exemplary density reducing agent include expandable thermoplastic microspheres comprising a thermoplastic polymer shell and a propellant entrapped therein are commercially available under the trademark EXPANCEL®. In one embodiment, the level of density reducing agents is from 0.01% to about 6%, or from about 0.1% to about 3%, or from about 1.5% to about 2.5%, or about 2%.

### Optional Ingredients

The shave preparations can further comprise additional optional ingredients. Suitable additional optional ingredients include perfume, preservatives, chelants, sensates (e.g. menthol), desquamation actives, anti-acne actives, anti-wrinkle/anti-atrophy actives, anti-oxidants/radical scavengers, flavonoids, anti-inflammatory agents, anti-cellulite agents, topical anesthetics, tanning actives, skin lightening agents, skin soothing and healing actives, antimicrobial actives, sunscreen actives, visual skin enhancers, humectants and moisturizing agents (e.g., glycerin, glycols, sorbitol) and the like. Such optional ingredients are described more fully in U.S. application Ser. No. 11/367,918, filed Mar. 3, 2006. Preferred additional optional ingredients include salicylic acid, opacifiers (e.g. mica and titanium dioxide), perfume, hydrophilic conditioning agents (e.g., glycerin) and skin sensates (e.g. menthol).

The shave preparations of the present invention may contain salicylic acid, its isomers, tautomers, salts and derivatives thereof. Alternatively, the compositions comprise from about 0.001% to about 5% salicylic acid. Alternatively, the compositions comprise from about 0.01% to about 2% salicylic acid. Alternatively, the compositions comprise from about 0.1% to about 1% salicylic acid. Without wishing to be bound by theory, it is believed that salicylic acid is efficacious for the treatment of acne on the skin. Moreover, the salicylic acid is capable of treating and/or reducing the presence of acne on the skin. Such treatment with the shave preparation of this invention involves applying the shave preparation to the skin and shaving the skin that has been treated with the shave preparation. Dermatologically acceptable salts include alkali metal salts, such as sodium and potassium; alkaline earth metal salts, such as calcium and magnesium; non-toxic heavy metal salts; ammonium and trialkylammonium salts such as trimethylammonium and triethylammonium. Derivatives of salicylic acid include, but are not limited to, any compounds wherein the CH3 groups are individually or in combination replaced by amides, esters, amino groups, alkyls, and alcohol esters. Tautomers of salicylic acid are the isomers of salicylic acid which can change into one another with ease so that they ordinarily exist in equilibrium. Thus, tautomers of salicylic acid can be described as having the chemical formula C7H6O3 and generally having a similar structure to salicylic acid.

The compositions of the present invention may include from about 0.001% to about 5%, alternatively from about 0.01% to about 2%, and alternatively from about 0.1% to about 1%, of alpha- or beta-hydroxy acids, and derivatives, salts, isomers and tautomers thereof. Non-limiting examples of alpha- and beta-hydroxy acids include alpha-hydroxy-butyric acid, alpha-hydroxyisobutyric acid, alpha-hydroxyisocaproic acid, alpha-hydroxyisovaleric, atrolactic acid, beta-hydroxybutyric acid, beta-phenyl lactic acid, beta-phenylpyruvic acid, citric acid ethyl pyruvate, galacturonic acid, glucoheptonic acid, glucoheptono 1,4-lactone, gluconic acid, gluconolactone glucuronic acid, glucuronolactone, glycolic acid, isopropyl pyruvate, lactic acid, malic acid, amndelic acid, emthyl pyruvate, mucic acid, pyruvic acid, saccharic acid, saccharic acid 1,4-lactone, tartaric acid and tartronic acid, and mixtures thereof.

For shaving preparations that do not produce enough visual contrast between areas of the skin that have and have not been shaved, opacifiers may be added to the shaving preparation. Opacifiers may be either inorganic or organic compounds. Inorganic opacifiers include, for example, titanium dioxide, zinc oxide, talc, mica or coated mica (with oxides of titanium, tin, or iron or bismuth oxychloride), magnesium aluminum silicate, bismuth oxychloride, or other minerals. These compounds can be added as powders, dispersions, or complexes. Organic opacifiers include, for example, opaque emulsions (e.g., containing Styrene/PVP copolymer, vinyl polymers, or latexes), metal salts of amines containing 14-20 carbon atoms per molecule, alkanolamides containing 14-20 carbon atoms per molecule, organic alcohols containing 14-20 carbon atoms per molecule, insoluble salts of stearic acid, glycol mono- or distearates, propylene glycol and glycerol monostearates and palmitates. Combinations of these opacifiers can also be used. The opacifying additive is typically included in an amount of about 1 to about 6%, preferably about 2 to about 5%, by weight of the composition.

The present composition comprises less than about 2% of an anionic surfactant and/or soap, or less than 1% anionic surfactant, or less than 0.5% anionic surfactant, or even free or substantially free of said anionic surfactant and/or soaps. Further, the present invention may contain up to about 5% of one or more lathering surfactants, or less than about 3%, or less than about 2%, or less than about 1.5%, or less than about 1%, or less than about 0.5% so long as the anionic surfactant component of the mixture is less than about 2%. In another embodiment, the present composition is free or substantially free of lathering surfactants. A lathering surfactant is defined herein as surfactant, which when combined with water and mechanically agitated generates a foam or lather. Lathering surfactants include those selected from the group consisting of anionic lathering surfactants, amphoteric lathering surfactants, and mixtures thereof. Generally, the lathering surfactants are fairly water soluble. Examples of anionic lathering surfactants are disclosed in McCutcheon's, Detergents and Emulsifiers, North American edition (1986), published by allured Publishing Corporation; McCutcheon's, Functional Materials, North American Edition (1992); and U.S. Pat. No. 3,929,678. A wide variety of anionic lathering surfactants are useful herein. Non-limiting examples of anionic lathering surfactants include those selected from the group consisting of sarcosinates, sulfates, sulfonates, isethionates, taurates, phosphates, lactylates, glutamates, and mixtures thereof. Other anionic materials useful herein are soaps (i.e., alkali metal salts, e.g., sodium or potassium salts) of fatty acids, typically having from about 8 to about 24 carbon atoms, preferably from about 10 to about 20 carbon atoms, monoalkyl, dialkyl, and trialkylphosphate salts, alkanoyl sarcosinates. Examples of zwitterionic or amphoteric surfactants are described in U.S. Pat. Nos. 5,104,646 and 5,106,609.

### Product Forms and Uses

The personal care compositions of the present invention can be used for as a hair removal preparation such as a post foaming shave gel. The present composition may be formulated as an aerosol foam, a post-foaming gel (which is the preferred form) or a non-aerosol gel or lather. It may be packaged in any suitable dispenser normally used for dispensing personal care compositions (such as shaving compositions). These include collapsible tubes, pump or squeeze containers, and aerosol-type dispensers, particularly those with a barrier to separate the post foaming gel composition from the propellant required for expulsion.

The latter type of dispensers include: (1) mechanically pressurized bag-in-sleeve systems in which a thin-walled inner bag containing the product is surrounded by an outer elastic sleeve that is expanded during the product filling process and provides dispensing power to expel the product (e.g., the ATMOS System available commercially from the Exxel Container Co.); (2) (a) a container preform comprising a polymeric preform and an elastically deformable band surrounding at least a portion of the polymeric perform such as described in U.S. 2009/0263174 to Chan et al; (3) manually activated air pump spray devices in which a pump system is integrated into the container to allow the user to pressurize the container with air in order to expel the product (e.g., the “AIRSPRAY” system available from Airspray International); (4) piston barrier systems in which the product is separated from the driving means by a tight-fitting piston which seals to the side of the container and may be driven by a spring under tension, by a vacuum on the product side of the piston, by finger pressure, by gas pressure to the piston, or by a variety of other means known to the packaging industry; and (5) bag-in-can (SEPRO) systems in which the product is contained in a flexible bag within a can, with a suitable propellant injected into the space between the can and the flexible bag. It is preferred to protect the composition from oxidation and heavy metal contamination. This can be achieved, for example, by purging the composition and container with nitrogen to remove oxygen and by utilizing inert containers (e.g., plastic bottles or bags, aluminum cans or polymer coated or lined cans).

The present composition can be used in combination with various hair removal applications (prior to, concurrently with, and/or after), including but not limited to shaving (wet or dry shaving, via electric razors, via powered or manual razors which can be reuseable or disposable, and combinations thereof), epilation, electrolysis, wax or depilatories as well as energy delivery devices to help regulate hair growth. Nonlimiting examples of energy deliver devices include: light, heat, sound (including ultrasonic waves and radio frequency), electrical energy, magnetic energy, electromagnetic energy (including radiofrequency waves and microwaves), and combinations thereof. The light energy may be delivered by devices including, but not limited to, lasers, diode lasers, diode laser bars, diode laser arrays, flash lamps, intense pulsed light (IPL) sources, and combinations thereof. See e.g. US2006/0235370A1.

### EXAMPLES

The following examples are provided to further illustrate exemplary shave preparations of the present invention. Material percentages are added neat. Activity levels are specified as needed.

*Percentages in Table 1 (Examples 1-10) and Table 2 (Examples 11-15) are on a weight by weight basis.

TABLE 1*

| | Concentrate | | | | | | | | | |
|---|---|---|---|---|---|---|---|---|---|---|
| Ingredient | Ex. 1 | Ex. 2 | Ex. 3 | Ex. 4 | Ex. 5 | Ex. 6 | Ex. 7 | Ex. 8 | Ex. 9 | Ex. 10 |
| Water | 86.9 | 85.24 | 84.89 | 83.3 | 84.89 | 82.83 | 80.74 | 78.9 | 79.77 | 73.77 |
| Cetyl Alcohol | — | — | — | — | 3 | 3 | 3 | 3 | 3 | 3 |
| Sepigel 305 (Polyacrylamide | 2 | 2 | 2 | 2 | 1 | 1 | 1 | 1 | 2 | 2 |
| & C13-14 Isoparaffin & Laureth-7) | | | | | | | | | | |
| Polyox N12K (PEG-23M) | 0.4 | 0.6 | 0.8 | 0.8 | 0.8 | 0.8 | 0.8 | 0.8 | 0.8 | 0.8 |
| Natrosol 250 HHR (HEC) | 0.8 | 0.8 | 0.8 | 0.8 | 0.8 | 0.8 | 0.8 | 0.8 | 0.8 | 0.8 |
| Glycerin | — | — | — | — | — | 2 | — | 2 | — | — |
| Brij 35 (Laureth-23, 100% Active) | 2 | — | — | — | — | — | — | — | — | — |
| Salicylic Acid | 0.5 | — | — | — | — | — | — | — | — | — |
| Plantaren 1200 N UP | — | 4 | 4 | 8 | 4 | 4 | 8 | 8 | 1 | 1 |
| (Lauryl Glucoside, 50% Active) | | | | | | | | | | |
| Lenocare SMS-30 (Sodium Myristoyl | — | — | — | — | — | — | — | — | 7 | 7 |
| Sarcosinate, 30% Active) | | | | | | | | | | |
| Mackam DAB-ULS (Lauramidopropyl | — | — | — | — | — | — | — | — | — | 6 |
| Betaine, 35% Active) | | | | | | | | | | |
| Sodium Lauryl Sulfate (29% Active) | — | — | — | — | 0.5 | 0.5 | 0.5 | 0.5 | 0.5 | 0.5 |
| Arlamol E (PPG-15 Stearyl Ether) | 2 | 2 | 2 | 2 | 2 | 2 | 2 | | 2 | 2 |
| Petrolatum | | — | — | — | — | — | — | 2 | — | — |
| Acusol OP301 Opacifier (40% solids) | 2.5 | 2.5 | 2.5 | — | — | — | — | — | — | — |
| (Water and Styrene/Acrylic Copolymer) | | | | | | | | | | |
| Expancel 920-WE40 D24 | 2 | 2 | 2 | 2 | 2 | 2 | 2 | 2 | 2 | 2 |
| Citric Acid | | 0.06 | 0.06 | 0.15 | 0.06 | 0.12 | 0.2 | 0.2 | 0.18 | 0.18 |
| Menthol | | — | 0.05 | 0.05 | 0.05 | 0.05 | 0.065 | — | 0.05 | 0.05 |
| Perfume | 0.5 | 0.5 | 0.6 | 0.6 | 0.6 | 0.6 | 0.6 | 0.5 | 0.6 | 0.6 |
| Glydant Plus | 0.4 | 0.3 | 0.3 | 0.3 | 0.3 | 0.3 | 0.3 | 0.3 | 0.3 | 0.3 |
| Volatile Self-foaming Agent | 2 | 2 | 2 | 2 | 1.5 | 1.5 | 2 | 1.5 | 1.5 | 1.5 |
| Elastic Modulus as measured | 154 | 237 | 226 | 225 | 529 | 271 | 227 | 218 | 231 | 139 |
| by Prep Rheometry Test Method | | | | | | | | | | |

TABLE 2*

| Concentrate | | | | | |
|---|---|---|---|---|---|
| Ingredient | Ex. 11 | Ex. 12 | Ex. 13 | Ex. 14 | Ex. 15 |
| Water | 87.94 | 76.9 | 75.37 | 80.75 | 80.735 |
| Cetyl Alcohol | — | 3 | 6 | 3 | — |
| Cetearyl Alcohol | — | — | — | — | 3 |
| (70/30) | | | | | |
| Sepigel 305 | 2 | 1 | — | 1 | 1 |
| (Polyacrylamide & | | | | | |
| C13-14 Isoparaffin & | | | | | |
| Laureth-7) | | | | | |
| Polyox N12K (PEG- | 0.3 | 0.8 | 0.8 | 0.8 | 0.8 |
| 23M) | | | | | |
| Natrosol 250 HHR | 0.8 | 0.8 | 0.8 | 0.8 | 0.8 |
| (HEC) | | | | | |
| Glycerin | — | 2 | 2 | — | — |
| Brij 35 (Laureth-23, | — | — | 1 | — | — |
| 100% Active) | | | | | |
| Plantaren 1200 N UP | 4 | 8 | 8 | 8 | 8 |
| (Lauryl Glucoside, | | | | | |
| 50% Active) | | | | | |
| Sodium Lauryl | — | 0.5 | 1 | 0.5 | 0.5 |
| Sulfate (29% Active) | | | | | |
| Arlamol E (PPG-15 | 2 | 2 | 2 | | 2 |
| Stearyl Ether) | | | | | |
| Isopropyl Isostearate | — | — | — | 2 | — |
| EGDS | — | 2 | — | — | — |
| Expancel 920-WE40 | 2 | 2 | 2 | 2 | 2 |
| D24 | | | | | |
| Citric Acid | 0.06 | 0.2 | — | 0.2 | 0.2 |
| Menthol | — | — | — | 0.05 | 0.065 |
| Perfume | 0.6 | 0.5 | 0.5 | 0.6 | 0.6 |
| Glydant Plus | 0.3 | 0.3 | — | 0.3 | 0.3 |
| Kathon CG | — | — | 0.03 | — | — |
| Phenoxyethanol | — | — | 0.5 | — | — |
| Volatile Self- | 2 | 1.5 | 1.5 | 1.5 | 1.5 |
| foaming Agent | | | | | |
| Elastic Modulus as | 384 | 417 | 233 | 196 | 168 |
| measured by Prep | | | | | |
| Rheometry Test | | | | | |
| Method | | | | | |

The above Examples are made according to the method below.
1. Weigh out the water in a vessel sufficient to hold the entire batch
2. Insert an overhead mixer with impeller into the vessel and increase agitation to create a vortex
3. Pre-blend the thickener and polymer powders
4. Sprinkle the polymer blend into the vortex until incorporated
5. Begin heating batch to 70 C to hydrate the polymers
6. Once the batch is at 70 C, add the oil and mix until uniform and dispersed
7. Add the liquid dispersion polymer to the batch and mix until uniform and hydrated, increasing rpms to maintain good mixing
8. Add the surfactant and mix until uniform and dispersed
9. Begin cooling batch to below 45 C
10. Once below 45 C, add the perfume, preservatives and other temperature-sensitive additives
11. Cool to below 35 C and QS with water
12. Blend the concentrate with the Volatile Self-Foaming Agent in the ratios described in the table and fill into pressurized container.

For product with acne control actives, weigh out the solubilizing agent and salicylic acid in a separate vessel and mix until dissolved. Add to the batch during STEP 6 addition.

For product with water-soluble actives, add to the batch during STEP 10 additions

For product with oil-soluble actives, add to the batch during STEP 6 additions

For product with improved tracking, add the opacifier during the STEP 10 additions

For product with lower density, add the Expancel to the batch during STEP 6 additions

While the specification concludes with the claims particularly pointing and distinctly claiming the invention, it is believed that the present invention will be better understood from the following description.

The devices, apparatuses, methods, components, and/or compositions of the present invention can include, consist essentially of, or consist of, the components of the present invention as well as other ingredients described herein. As used herein, “consisting essentially of” means that the devices, apparatuses, methods, components, and/or compositions may include additional ingredients, but only if the additional ingredients do not materially alter the basic and novel characteristics of the claimed devices, apparatuses, methods, components, and/or compositions.

All percentages and ratios used herein are by weight of the total composition and all measurements made are at 25° C., unless otherwise designated. A degree is a planar unit of angular measure equal in magnitude to 1/360 of a complete revolution.

All measurements used herein are in metric units unless otherwise specified.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A shave preparation comprising:
water;
one or more lipophilic skin conditioning agents;
one or more thickening agents;
one or more emulsifying agents; and
one or more lubricants
wherein the composition is in the form of a self-foaming cream or lotion having an elastic modulus (G′) of about 150 Pa to about 1000 Pa measured with the Prep Rheology Test Method defined herein and contains less than 2% anionic surfactant and is substantially free of soap.

2. The shave preparation of claim 1, having an elastic modulus of from about 150 Pa to about 550 Pa.

3. The shave preparation of claim 2, wherein said thickening agent comprises at least one of a carboxylic acid polymers, polyacrylamide polymers, polysaccharides, and mixtures thereof.

4. The shave preparation of claim 3, wherein the level of thickening agent is from about 0.1% to about 5%.

5. The shave preparation of claim 4, wherein the shave preparation is substantially free of a gel network.

6. The shave preparation of claim 1, wherein the shave preparation comprises from about 1% to about 12% by weight of the one or more lipophilic skin conditioning agents.

7. The shave preparation of claim 1, wherein the shave preparation comprises from about 0.1% to about 5% by weight of the one or more thickening agents.

8. The shave preparation of claim 1, wherein the shave preparation comprises from about 0.1% to about 8% by weight of the one or more emulsifying agents.

9. The shave preparation of claim 1, wherein the shave preparation comprises from about 0.1% to about 8% by weight of the one or more lubricants.

10. The shave preparation of claim 1, further comprising one or more of a salicylic acid, an opacifier, and a combination thereof.

11. The shave preparation of claim 10, wherein the shave preparation comprises from about 0.001% to about 5% by weight of the salicylic acid.

12. The shave preparation of claim 10, wherein the shave preparation comprises from about 1 to about 6%, by weight of the opacifier.

13. The shave preparation of claim 1, substantially free of a cationic skin conditioning agent.

14. The shave preparation of claim 1, comprising less than 2% of a lathering surfactant.

15. The shave preparation of claim 1, comprising less than about 6% of a gel network.

16. The shave preparation of claim 1, further comprising a density reducing agent.

17. A shave preparation comprising:
at least 60% water;
from about 2% to about 4% of one or more lipophilic skin conditioning agents;
from about 0.25% to about 3% of one or more thickening agents;
from about 0.25% to about 3% of one or more emulsifying agents; and
from about 0.25% to about 3% of one or more lubricants;
wherein the composition is in the form of a self-foaming cream or lotion having an elastic modulus (G′) of about 150 Pa to about 1000 Pa measured with the Prep Rheology Test Method defined herein and contains less than 2% anionic surfactant and is substantially free of soap and is substantially free of a gel network.

18. The shave preparation of claim 17, wherein the shave preparation comprises from about 0.001% to about 5% by weight of the salicylic acid.

19. The shave preparation of claim 17, further comprising a density reducing agent.

20. A method of treating acne on skin comprising applying the shave preparation of claim 1 to the skin and shaving the skin.
